(19) United States
(12) Patent Application Publication
(10) Pub. No.: US 2023/0008747 A1
(43) Pub. Date: Jan. 12, 2023

(54) Warming Sleeve

(71) Applicant: Stephanie Noyes, Falmouth, ME (US)

(72) Inventor: Stephanie Noyes, Falmouth, ME (US)

(21) Appl. No.: 17/369,363

(22) Filed: Jul. 7, 2021

(51) Int. Cl. A41D 13/005; H05B 3/34

(52) U.S. Cl. CPC A41D 13/0051; H05B 3/342; A41D 2400/12

(57) ABSTRACT

An electrically powered warming sleeve comprised of multiple fabric layers enclosing one or more planar heating panels arranged for positioning the panels over major muscle groups. Operation of the warming sleeve is controlled by a switch, preferably with multiple “on” positions to adjust the level of heating. The switch is conveniently accessible on the exterior of the sleeve and may be made integral with LED indicators to signal the active level of heating. In some embodiments, a receptacle positioned at the proximate posterior portion of a sleeve houses a power receiving means which may be electrically connected to a rechargeable battery. Preferably, the heating panels of the sleeve are engineered to eliminate hot spots and incorporate material to emit far infrared radiation that will penetrate soft tissues of a wearer. The warming sleeve may be used to warm extremities while maintaining dexterity of the digits or as a therapeutic device.

### TECHNICAL FIELD

The present invention relates generally to powered heating devices worn on the person, and more specifically to a portable heating sleeve for warming a forearm and hand.

### BACKGROUND

Exposure to low temperatures can be uncomfortable and even dangerous for persons without adequate protection from the cold. As a result of the body's natural response to maintain core temperature and limit peripheral blood flow, extremities such as hands and feet are most affected. Garments worn to combat the cold are often heavy, bulky, and restrictive to movement. Gloves and wraps succeed in keeping hands and fingers warm but typically must be removed to complete tasks requiring significant facility of the hands. In the coldest environments, feet and hands eventually numb and dexterity of fingers becomes greatly impaired.

Accordingly, there is a need for a device to maintain warmth in the hands and fingers while still maintaining a high degree of manual dexterity. Embodiments of the disclosed warming sleeves maintain warmth in the hands and forearms of a wearer while leaving the fingers exposed. Worn in cold temperatures, the novel device increases circulation of warmed blood to the fingers, providing deep internal warmth in a manner that is superior to wearing gloves.

### SUMMARY

Warming sleeves of the present invention comprise a tubular body sized to cover at least the majority of a person's forearm. The body of the sleeve comprises multiple layers, at least one layer of which is a stretch fabric, providing compression to the forearm and keeping the sleeve in place. One or more heating panels within the sleeve is positioned to be in close proximity to soft tissues of the forearm, warming blood as it is circulated toward the hands. The device receives power from a power source and applies the power to a heating circuit having a switch. The switch applies or holds back power from one or more heating panels. When power is applied to the heating panels, the forearm is warmed from the heat of the sleeve. Blood being pumped away from the heart moves through the soft tissues of the warmed forearm and travels into the hands and digits, bringing warmth to the distal portion of the limb.

In preferred warming sleeve embodiments of the present invention, a planar heating panel is comprised of a graphitized heating element within a protective, electrically insulating envelope. In some embodiments, a heating element within a heating panel has apertures for reducing hot spots that would otherwise be produced by the heating element. In further preferred embodiments, the heating panel also comprises a heat spreader component in contact with or in close proximity to the heating element to help dispense or better apportion heat throughout the heating panel. In still another embodiment, a heating panel further comprises a matrix or fabric impregnated with ceramic particles known to emit far infrared radiation when thermal energy is absorbed.

Preferred embodiments of warming sleeves of the present invention comprise tubular bodies that extend distally past the forearm to cover the palm and posterior surface of a hand while still leaving digits of the hand exposed. In some embodiments, a round opening or slit is provided laterally at the distal end of an extended sleeve body so that a thumb may protrude. In these extended body embodiments, an additional heating panel may be included to warm the posterior surface of the hand.

Some preferred embodiments of a sleeve of the present invention include electronic components assembled to provide variable average currents or duty cycles to one or more heating panels. Different duty cycles are selected using a switch having a plurality of “On” modes and a single “Off” mode for applying the particular duty cycle desired. In a preferred embodiment, power from a battery is pulse width modulated using a microcontroller and delivered through insulated conductors in the sleeve to groups of heating panels at the same time. In further preferred embodiments, the different groups of heating panels are simultaneously provided with different average currents or duty cycles while functioning together in the same mode.

### DESCRIPTION OF THE DRAWINGS

FIG. 1 is a diagrammatic view of a warming sleeve showing a heating panel positioned between compression fabric layers to heat the anterior forearm of a wearer.

FIG. 2 shows the heating circuit of the warming sleeve of FIG. 1.

FIG. 3 is a diagrammatic posterior view of a warming sleeve with multiple heating panels.

FIG. 4 is a diagrammatic view the anterior side of the warming sleeve of FIG. 3.

### DETAILED DESCRIPTION

The powered warming sleeves of the present invention provide warmth to the digits of the hand by encouraging circulation and warming blood as it moves through a wearer's forearm. Embodiments of the sleeve may be worn alone, without any covering, to free the wearer's digits from hindrances that would inhibit tactile dexterity. When dexterity of the fingers is not the primary aim, the sleeves may be worn covered by gloves or similar heat retaining garb to maximize the retention of heat provided by the sleeve.

Warming sleeves of the present invention comprise multilayered, tubular, fabric bodies having at least one layer of a compression fabric and at least one heating panel. Heating panels may be fixed to the body of a sleeve by various means including sewing and gluing etc., or the panels may be fixed in place to the body of the sleeve by the clamping force from a compressive fabric layer. In some preferred embodiments, heating panels are sandwiched between layers of compressive fabric sewn together to encompass the panels and keep them positioned over significant physiologic areas in close proximity to the targeted tissues of the wearer.

Heat is generated in warming sleeves of the present invention by electrically powering one or more of the heating panels. The heating panels are relatively planar and may simply comprise a resistive heating element in a protective, electrically insulating envelope that will lay closely to soft tissues for warming. When powered, the elements of the heating panels provide thermal radiation. In preferred embodiments, the heating panels are thin, flexible, and comprise a heating element impregnated or woven with or otherwise in proximity to far infrared emitting materials, such as powdered graphite, tourmaline or other ceramics to produce far infrared radiation (FIR) that penetrates and is absorbed by soft tissues.

Power to the heating panels is controlled by a switch and may be provided by various power sources. In some preferred embodiments, warming sleeves comprise a power receiving means in the form of a battery holder that electrically communicates with heating panels while controlled by a switch. The switch and battery are preferably positioned along the sleeve at locations that are unobtrusive to a wearer's movement and that complement the operation and function of the sleeve. Ideally, the battery is placed at the proximate posterior of the sleeve, gaining significant warmth from the forearm and enhancing battery function. The switch, which may include light emitting diode (LED) indicators, is best positioned at the anterior wrist area to facilitate ease of access to the switch control and a good view of the mode of operation being signaled by the LEDs.

In some embodiments, warming sleeves of the present invention may be worn as a therapeutic device, providing compressive force and warmth to tissues of a wearer. In preferred therapeutic embodiments, far infrared emitting ceramic particles are incorporated into components of the warming sleeve to enhance deep tissue warming and provide an even greater reparative effect.

The following descriptions of particular embodiments refer to accompanying drawings. The same reference numbers in different drawings identify the same or similar elements. The language, examples, and embodiments shown and/or described herein are provided for description and not limitation. Rather than being limited to the details of the particular arrangements shown, the present warming sleeve invention is to be understood to include the many combinations of features and/or elements referenced herein in keeping with the spirit and scope of the present disclosure.

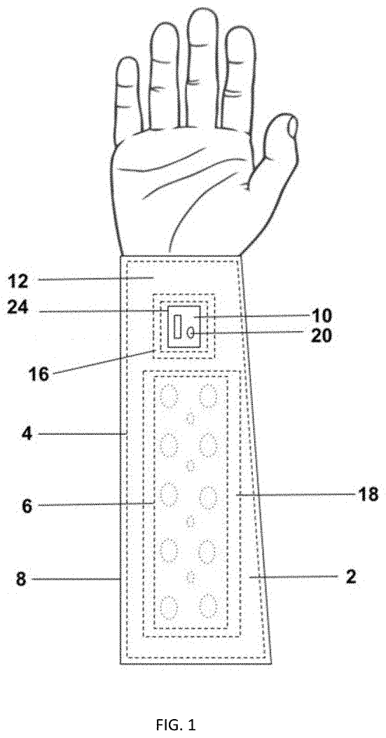

Shown in FIG. 1, a warming sleeve comprises a tubular fabric body 2 that is sized to cover a portion of a forearm between a hand and elbow of a wearer. The body 2 is comprised of multiple, tubular layers of fabric, concentrically aligned. A first layer 4 of the sleeve body is an elastic compression fabric designed to be in direct contact with the forearm surface of the wearer. Above or external to the first layer 4 is a heating panel 6. The heating panel 6 is situated over the anterior forearm adjacent to large muscle groups with significant vascularization. Above or external to the heating panel 6 is a second layer 8 of fabric concentrically aligned with the first layer 4. The first layer 4 and the second layer 8 sandwich the heating panel 6 in between. Also sandwiched between the first layer 4 and the second layer 8 of the body is an On/Off switch 10 positioned at the anterior wrist portion 12 of the sleeve. A plastic battery holder, adapted to receive power from a DC battery, is housed within an external receptacle compartment sewn onto the exterior of the second layer on the proximate posterior surface of the sleeve. (Not shown) The battery holder is configured to make electrical contact with a 9-volt battery and is electrically connected to the heating panel 6 through the switch 10.

Referring still to FIG. 1, the first layer 4 and second layer 8 of the body of the sleeve are sewn together sufficiently to remain aligned and to form compartments for securing the heating panel 6 and the switch 10. A switch compartment 16 and a heating panel compartment 18 are formed between the layers to minimize movement of the heating panel 6 and the switch 10 within the sleeve.

The switch 10 includes a single light emitting diode (LED) 20 on its face that illuminates when the switch is closed and power from a battery is delivered to the heating panel 6. Insulated conductors running between the layers of the sleeve electrically connect the heating panel 6 with the switch 10 and the battery holder. Current flows through the heating panel 6 when the switch 10 is set to its “On” position and current is stopped from flowing when the switch is set to its “Off” position. Both the LED 20 and the switch 10 are exposed through an opening 24 defined by the second layer 8 of the sleeve body.

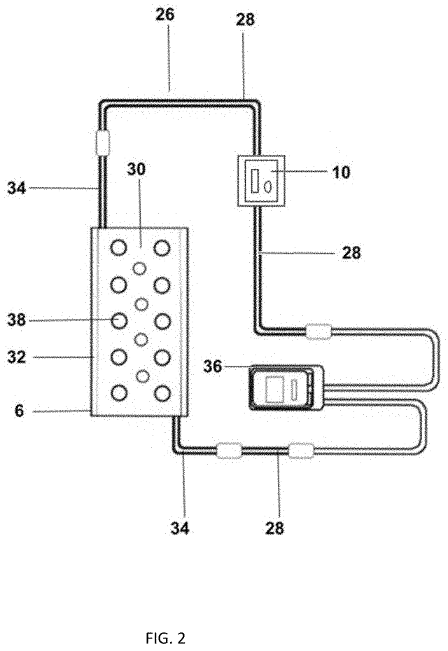

FIG. 2 is a more complete view of the heating circuit 26 of the warming sleeve of FIG. 1 showing the components and conductors 28. The heating circuit 26 includes a heating panel 6 comprised of a resistive heating element 30 enclosed within a planar, protective, and electrically insulating envelope 32. Lying flat, the heating panel 6 is approximately 3 inches wide by 6 inches long and 5/16 of an inch thick.

Covered by the protective envelope 32 of the heating panel 6, the heating element 30 is comprised of a carbon particle matrix which emits radiation in the far infrared range. Electrical power is moved through the heating element 30 via a pair of lead wires 34, a.k.a. conductors, electrically communicating with the heating element 30. The lead wires 34 extend though the protective envelope 32 and form part of the heating circuit 26 of the sleeve. As shown in FIG. 2, one of the heating element lead wires is electrically connected to the switch side of the heating circuit and the other heating element lead wire electrically communicates with a terminal of the power source through the battery holder that serves as a power receiving means 36. The switch 10 communicates with the opposite terminal of the power source through the power receiving means 36. When set in the “on” position, the switch 10 is closed and completes the heating circuit, allowing power to flow through the heating element.

The planar heating element 30 is perforated with multiple apertures 38 that are filled with protective, electrically insulating material continuous with the envelope 32. The apertures 38 are organized in three columns along the length of the heating element. The first column and the third column comprise five round apertures having a diameter of 0.5 inches. Staggered in between the first and third column is a second column of four round apertures having a diameter of 0.375 inches.

The pattern of apertures 38 of the heating element 30 directs and distributes current flow through the element and is optimized to increase energy efficiency and provide for even heating. The aperture pattern is designed in consideration of the size of the heating element, the voltage of the power source, and the thickness and density of layers between the wearer and the heating element so that the apertures are sized and spaced to prevent the concentration of energy in any area of the heating panel 6. The heating panel 6 is also protected from overheating by inherent properties of the heating element. As the temperature of the panel rises, electrical resistance in the heating element increases, thereby reducing current flow and maintaining a consistent temperature in the sleeve. Having the size, shape, placement, and quantity of apertures selected to eliminate hot spots and a heating element regulating the power that it receives, no additional protective measure is needed in the heating circuit of the sleeve. Heating panels of this type may be obtained from EXO2, McDonough, Ga.

In another embodiment, a heating panel of a warming sleeve includes a material having high thermal conductivity in contact with or in close proximity to the heating element, functioning to distribute heat from the heating element more evenly throughout the heating panel. In another embodiment, a heating panel of a warming sleeve includes a composite heating element having a heat spreader comprising a thin, flexible graphitized polymer layer in thermal communication with the heating element. In still another embodiment, a heating panel includes a heat spreader comprising graphite particles in direct contact with the heating element.

Warming sleeves of the present invention may have heating elements with aperture patterns varying in size, shape, quantity, and distribution of apertures for different heating panels. In some embodiments, a heating panel may have a heating element with many more apertures to draw less current or without any apertures or may have a thicker protective envelope in certain areas of the panel to shield a wearer from elevated temperatures.

Protective envelopes of heating panels for the present invention are preferably made from suitable electrically insulating material. Preferably, the material is flexible so that it may easily conform to a wearer's anatomy. The protective envelopes may be made thin to reduce heat absorption or made thicker to retain heat instead. Materials for protective envelopes are preferably also water resistant and include rubbers and plastics such as polychloroprene, nitrile butadiene, polyurethane, and others.

In a preferred embodiment of a warming sleeve according to the present invention, a power receiving means comprises half of a two-wire electrical connector instead of a battery holder. The warming sleeve half of the connector is permanently wired to a heating panel circuit of the warming sleeve and removably attaches to a mating half of the two-wire connector, which is electrically wired to a multicell battery pack. With both halves of the two-wire connector attached together, voltage from the battery pack is provided to the heating panel circuit when the switch is “on”.

In a preferred embodiment, a warming sleeve receptacle is a closeable pouch situated at the proximate posterior area of the sleeve. The pouch is designed to house a multicell battery pack along with a two-wire connector and the associated wiring. In another embodiment, the receptacle houses a power receiving means comprising an electrical connector half that removably attaches to a power cord delivering DC power from a source that is external to the warming sleeve.

Batteries and battery packs powering warming sleeves of the present invention may be comprised of primary or secondary cells but are preferably batteries or battery packs of rechargeable Lithium ion cells. In a preferred embodiment having rechargeable batteries, a warming sleeve includes a charging circuit in electrical communication with a power receiving means. The charging circuit includes a jack or charging connector for inserting a charging cable. In another embodiment, a warming sleeve includes a 12-volt rechargeable battery pack permanently wired to a connector that removably attaches to a mating connector serving as a warming sleeve heating circuit power receiving means. The battery pack is charged by removing it from the power receiving means and electrically connecting the battery pack to a suitable charging apparatus.

Switches for circuits of warming sleeves of the present invention are preferably small and low-profile but may be any one of various types of switches such as; rocker switches, rotary switches, push button switches, slide button switches, etc., so long as the switch or switches chosen are easy to operate and have adequate capacity for the circuits to which they are connected. In a preferred embodiment, a warming sleeve heating circuit is operated by single push button switch having multiple “On” positions and a single “Off” position. The multiple “On” positions are used to select the level of heat provided by the heating panels. In an alternate embodiment, a heating circuit of a warming sleeve is controlled by two switches. The first switch operates to turn the heating circuit “On” and “Off” while the second switch is used to adjust the level of heat provided by the heating panels.

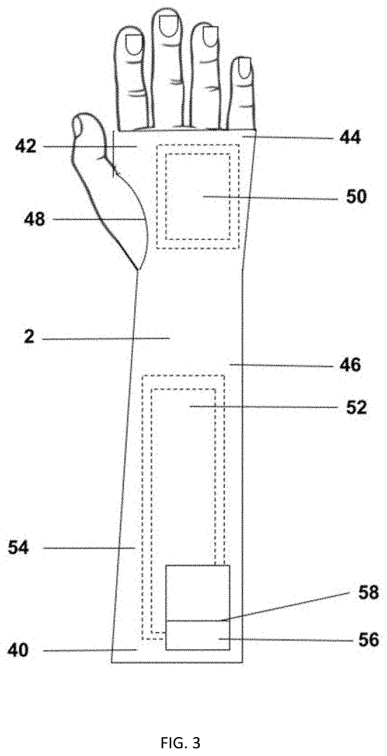

Warming sleeves of the present invention preferably include a heating circuit with multiple heating panels and electronic circuitry to direct power to the panels. Shown now in FIG. 3 is a diagrammatic representation of a warming sleeve fitted to the hand and forearm of a wearer. The warming sleeve is comprised of a tubular fabric body 2 having an open proximate end 40 and an open distal end 42. The body 2 is comprised of multiple fabric layers sewn together and concentrically aligned. Both a first layer and a second layer of the body are compression fabric, with the first layer being inside the second layer and the second layer of compression fabric being the outer layer 54.

In this embodiment, a tubular hand section 44 extends from a tubular forearm section 46 of the body 2. The body 2 is formed and sized to cover the forearm and the proximate portion of the hand while leaving the digits exposed. A thumb hole 48, defined by an opening in the fabrics of the hand section 44, is positioned laterally to allow the thumb of a wearer to protrude.

As can be seen from the posterior view of the warming sleeve shown in FIG. 3, a hand heating panel 50 is sewn between the first layer and second layer of the hand section. As with the heating panel of the embodiment shown in FIG. 1, the hand heating panel 50 is comprised of an internal heating element enclosed within a protective, electrically insulating envelope. (Not shown) The hand heating panel 50 is positioned within the hand section to be adjacent to posterior tissues of the hand.

Similarly, a posterior forearm heating panel 52 is sewn between the first layer and second layer of the forearm section 46 of the body, adjacent to soft tissues of the posterior forearm. The heating element of the posterior forearm heating panel 52 includes a pattern of apertures and is enveloped by a protective, electrically insulating material. However, the protective envelope of the posterior forearm heating panel is thinner and less heat absorbing than the envelope of the hand heating panel 50.

Sewn into the proximate posterior portion of the outer layer 54 is a receptacle 56 adapted to enclose a 7.4 volt, 3500 mAh lithium-ion battery or battery pack and a power receiving means. The receptacle 56 includes a lip and flip closure 58 that may be opened to access the battery and closed to the secure the battery in place.

Referring to other embodiments of the invention in general, an aesthetically pleasing outer shell layer may be adapted to cover a warming sleeve and made from a durable fabric, such as nylon or other tough fabrics, while other warming sleeves comprise only compression fabric layers and are equipped with no outer shell layer at all. Receptacles housing a battery and/or power receiving means may take the form of a pocket or pouch that is sewn, adhered, or otherwise fixed to the outer layer of the sleeve. The receptacle may be designed to remain open or may have any one of many other mechanical closures, such as a zipper, hook and loop, button, snap, elastic, or draw string etc.

Returning to the embodiment shown in FIG. 3, the receptacle 56 for the battery and power receiving means is placed on the proximal posterior-medial aspect of the forearm for optimal joint mobility and musculoskeletal function. In addition to being away from movement, the battery is in proximity to a large muscle group that generates body heat, which minimizes energy depletion of the battery in cold environments. In other embodiments, the receptacle for the battery and/or power receiving means is positioned elsewhere on the outermost layer, between the layers, or on the inside of the warming sleeve.

Still referring to the warming sleeve of FIG. 3, the heating panels and power receiving means are electrically connected as part of a heating circuit of the warming sleeve. Conductors running between the layers of the warming sleeve body electrically connect the heating circuit components. (Not shown)

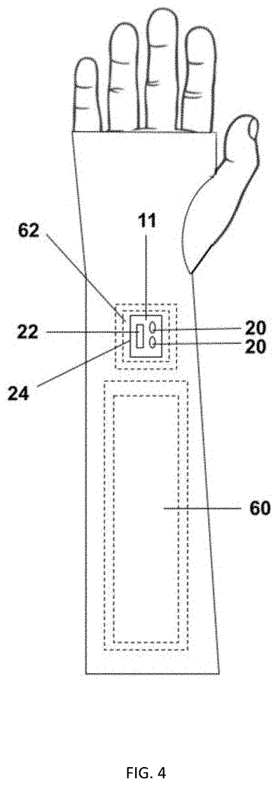

Also connected to the heating circuit but on the anterior portion of the warming sleeve is an anterior forearm heating panel, of similar construction to the posterior forearm heating panel, and a circuit board having a button switch with LEDs exposed to the outside of the sleeve. Shown now in FIG. 4 is a diagrammatic representation of the warming sleeve of FIG. 3 viewed from an anterior perspective. The anterior forearm heating panel 60 is positioned between the compression layers and confined by a stitched compartment to be adjacent to soft tissues of the anterior portion of the forearm. Further along the anterior portion of the warming sleeve and distal to the anterior forearm heating panel 60 is the button switch 11. The button switch 11 is integrated with a pair of LED indicators 20 and a circuit board 62 comprising a microcontroller circuit. The LED indicators 20 and button 22 of the switch 11 are visible and accessible through an opening 24 of the fabric layers. A remaining portion of the circuit board 62, not visible in the opening, is confined by a compartment defined by stitching of the fabric layers, keeping the button switch 11 and LEDs 20 optimally positioned in the sleeve.

The microcontroller circuit includes pulse width modulation (PWM) and relay functions. The button switch 11 and LEDs 20 are in electrical communication with a microcontroller on the circuit board 62 and together provide “Off” and “On” mode functions discernable by observing the LEDs 20. The “On” mode functions include a “Hi” mode and a “Low” mode, each indicated by a different LED. With the button switch set to one of the “On” positions, the circuit board 62 receives power from conductors in electrical communication with the power receiving means and relays current to the heating circuit of the sleeve according to the mode selected.

The heating panels of the warming sleeve of FIGS. 3 and 4 are wired in series and the level of heat produced by the sleeve is adjusted by modulating the pulse width, i.e., changing the duty cycle of current provided to the heating circuit as directed by the microcontroller. The single output PWM microcontroller provides duty cycles that correspond to the “Hi” position and the “Low” position settings of the button switch 11. The duty cycles produced by the microcontroller include two periods; a period during which current is actively applied to the heating circuit and a period of inactivity during which no current is applied. The ratio of active to inactive periods of the duty cycle of the “Hi” mode is approximately three times greater than the ratio of active to inactive periods of the duty cycle of the “Low” mode, with the “Hi” mode having a duty cycle of approximately 99% and the low mode having a duty cycle of approximately 33%.

Still referring to the warming sleeve of FIGS. 3 and 4, the duty cycle provided for the “Hi” mode is equivalently communicated to each of the heating panels. Similarly, the duty cycle provided for the “Low” mode is the same for all the heating panels of the heating circuit. Heat produced by the heating circuit may be balanced between the heating panels by including particular thicknesses for the protective envelopes, selecting specific aperture patterns for the heating elements, and/or employing heat spreaders in thermal communication with a heating element of any one or more of the heating panels. More specifically, the protective material forming the envelope covering the hand heating panel of the warming sleeve of FIGS. 3 and 4 is thicker and considerably more heat absorbing than the protective material of the posterior and anterior forearm heating panels. In addition, the heating element of the hand heating panel of the warming sleeve of FIGS. 3 and 4 has less than half the number of apertures than the heating elements of the posterior and anterior forearm heating panels.

In other embodiments of warming sleeves with multiple heating panels, heating panels of the heating circuit are wired in parallel loops and heat generated by the heating circuit is balanced by proportioning current between the loops of the heating circuit.

One such parallel loop heating panel embodiment of a warming sleeve comprises a PWM controller circuit that produces multiple concurrent outputs and multiple heating panels each electrically communicating with a distinct PWM controller output. Functioning in combination with a switch, the PWM controller provides a selection of “On” modes in addition to an “Off” mode. Each “On” mode corresponds with a particular set of duty cycle outputs that affects the multiple heating panels simultaneously. “On” mode sets with more active duty cycles, e.g., “Hi” mode, produce more heat from the heating circuit than “On” mode sets with less active duty cycles, e.g. “Low” mode.

To balance the heating circuit in this parallel loop embodiment, different “On” mode outputs are provided by the PWM controller to the individual heating panels for a given set. The duty cycle output for a heating panel positioned in a hand section of the warming sleeve is less active than the duty cycle outputs for larger heating panels positioned in a forearm section of the sleeve. In preferred embodiments of warming sleeves with multiple heating panels wired in parallel, the PWM controller is a microcontroller with multiple outputs that electrically communicates with a different solid-state relay for each of the heating panels. The microcontroller and relays function to provide multiple “On” modes with distinct duty cycles for different panels simultaneously.

In other embodiments of a warming sleeves with multiple heating panels, parallel heating panel loops include one or more resistors to limit the flow of current through the loops. Heat generated by the heating circuit in any one of several “on” modes is balanced between the loops by the particular ohm value of resistors selected for the loops. Depending on the size and type of a particular heating panel and where in the warming sleeve the heating panel will be placed, resistors may be selected to increase or decrease the proportion of heat produced by a specific heating panel loop with respect to the other loops of the heating circuit. In one embodiment, a thermistor is used in conjunction with resistors and is in electrical communication with the heating circuit to reduce or interrupt power when a specific temperature is reached.

Preferred embodiments of warming sleeves of the present invention comprise at least one compression fabric layer to help hold one or more heating panels in close proximity to soft tissues while also stimulating circulation. Some embodiments include a durable external shell layer opposite an inner lining that together sandwich compression fabric layers between. Although warming sleeve heating circuits may be designed to be inherently self-regulating, additional temperature controls such as thermistors and bimetal mechanical thermostats may be used with any embodiments of the present invention for additional safety.

Although the present invention has been described with reference to a few simplified embodiments, numerous modifications and variations can be made within the scope of the present disclosure. No limitation with respect to the specific embodiments disclosed herein is intended or should be inferred. Rather, it should be understood that the particular features and structures shown and/or described in drawings, specification, or appended claims may be combined in any suitable manner in keeping with the spirit and scope of the present disclosure.

We claim:

1. A warming sleeve comprising:
A tubular, fabric body formed and sized to cover a forearm, said fabric body having a proximate end and a distal end;
One or more heating panels fixed to the fabric body, said heating panels each having an internal heating element, said heating panels positioned to be adjacent to soft tissues of the forearm when the sleeve is worn;
An electrical switch having at least one “on” position and at least one “off” position;
A power receiving means for receiving and applying voltage from a power source; and
A plurality of conductors electrically connecting the power receiving means with the heating panels through the switch; whereby said conductors connect the power receiving means, the switch, and the heating panels to form a heating circuit, whereby voltage received by the power receiving means is applied to the heating panels when the switch is in the “on” position.

2. The warming sleeve of claim 1 wherein the tubular fabric body further comprises a tubular hand section at the distal end, said hand section extending the tubular fabric body and being formed and sized to cover a proximate portion of a hand while leaving digits of the hand exposed, said tubular hand section further comprising a heating panel positioned to be adjacent to posterior tissues of the hand, said heating panel being in electrical communication with the heating circuit.

3. The warming sleeve of claim 2 wherein the tubular body further comprises a slit or opening through the hand section, said slit or opening positioned laterally on the hand section for protrusion of a thumb.

4. The warming sleeve of claim 1 whereby the heating panels comprise an anterior forearm heating panel and a posterior forearm heating panel.

5. The warming sleeve of claim 1 wherein the tubular fabric body is comprised of a first layer and a second layer, both layers being tubular and concentrically aligned, said first layer positioned under the heating panels to make direct contact with the hand and forearm when the sleeve is worn.

6. The warming sleeve of claim 5 wherein the first layer or the second layer, or both, is comprised of a compression fabric.

7. The warming sleeve of claim 5 further comprising a receptacle containing the power receiving means.

8. The warming sleeve of claim 7 wherein the receptacle is fixed to the exterior of the fabric body and configured for containing a DC battery and power receiving means, said DC battery in electrical communication with the power receiving means.

9. The warming sleeve of claim 8 further comprising a charging circuit in electrical communication with the power receiving means, said charging circuit having a jack or charging connector for recharging a DC battery in electrical communication with the power receiving means.

10. The warming sleeve of claim 8 wherein the receptacle is fixed to the proximate end of the fabric body.

11. The warming sleeve of claim 1 further comprising a thermistor circuit in electrical communication with the heating circuit, said thermistor circuit configured to reduce or interrupt power to the heating panels when a specific temperature is reached.

12. The warming sleeve of claim 1 wherein at least one of the heating panels is comprised of a planar heating element with multiple apertures, whereby heat from the heating panel is directed and distributed to minimize hot spots.

13. The warming sleeve of claim 1 wherein the heating panels are comprised of graphite.

14. The warming sleeve of claim 1 further comprising a heat spreader in thermal communication with the heating element, whereby heat from the heating element is dispensed or apportioned to minimize hot spots.

15. The warming sleeve of claim 1 wherein at least one of the heating panels produces far infrared radiation when powered by the heating circuit.

16. The warming sleeve of claim 1 further comprising a microcontroller circuit in electrical communication with the heating circuit and wherein the switch has multiple “on” positions, said microcontroller circuit configured to affect a variety of duty cycles for powering the heating panels, said switch “on” positions selecting for a particular duty cycle mode.

17. The warming sleeve of claim 16 wherein the microcontroller circuit is further configured to produce distinct duty cycles for different heating panels simultaneously.

18. The warming sleeve of claim 16 further comprising one or more LEDs to indicate the status of particular “On” and “Off” modes, said LEDS being integrated with the switch and microcontroller circuit on a circuit board.

19. The warming sleeve of claim 18 wherein the circuit board is fixed to the sleeve at an anterior wrist position exposing the switch and LEDs outside the sleeve.

20. A warming sleeve comprising:
A tubular, fabric body formed and sized to cover a forearm and hand while leaving the digits exposed, said fabric body having an open proximate end and an open distal end, said fabric body having a forearm section and a hand section, said hand section having an anterior side and a posterior side and a lateral opening positioned for exposing a thumb;
One or more forearm heating panels fixed to the forearm section of the fabric body, said forearm heating panels positioned to be adjacent to soft tissues of a forearm when the sleeve is worn;
A hand heating panel fixed to the posterior side of the hand section, said hand heating panel positioned to be adjacent to posterior tissues of a hand when the sleeve is worn;
A planar, heating element in at least one of the heating panels, said heating element perforated with multiple apertures to minimize hot spots within the heating panel;
An electrical switch having multiple “on” positions and at least one “off” position;
A power receiving means for receiving and applying voltage from a power source;
A plurality of conductors electrically connecting the power receiving means with the heating panels through the switch; whereby said conductors connect the power receiving means, the switch, and the heating panels to form a heating circuit; and
A microcontroller circuit in electrical communication with and configured to control the heating circuit, said microcontroller circuit configured to affect multiple duty cycles in the heating panels, said duty cycles being selectable from the switch, whereby voltage received by the power receiving means is applied to the heating panels according to the microcontroller circuit when the switch is in one of the “on” positions.
